(19) United States
(12) Patent Application Publication
(10) Pub. No.: US 2008/0032024 A1
(43) Pub. Date: Feb. 7, 2008

(54) Maltogenic Alpha-Amylase Variants

(76) Inventors: Lars Beier, Lyngby (DK); Allan Svendsen, Horsholm (DK); Torben Vedel Borchert, Birkeroed (DK)

Correspondence Address: NOVOZYMES NORTH AMERICA, INC., 500 FIFTH AVENUE, SUITE 1600, NEW YORK, NY, 10110, US

(21) Appl. No.: 11/572,272

(22) PCT Filed: Jul. 22, 2005
(86) PCT No.: PCT/DK05/00506

§ 371 Date: Jan. 18, 2007

(30) Foreign Application Priority Data
Aug. 2, 2004 (DK) PA 2004 01173

(51) Int. Cl. C12N 9/28; A21D 10/00; C12N 15/56

(52) U.S. Cl. 426/549; 435/202; 536/23.2

(57) ABSTRACT

The inventors realized that in some applications the control of the maltose-to-glucose ratio is of great importance. Particularly for ethanol production from granular starch by fermentation, it may be an advantage to form a larger amount of glucose which is more readily fermentable than maltose. Particularly for production of maltose syrups glucose is an undesired product, and hence it of interest to increase the maltose-to-glucose ratio. They then developed a method of constructing such variants of based on the three-dimensional structure of a parent maltogenic alpha-amylase.

### FIELD OF THE INVENTION

The invention relates to the preparation of variants of a parent maltogenic alpha-amylase, where hydrolysis products of said variants having a modified of maltose-to-glucose ratio as compared to hydrolysis products of the parent maltogenic alpha-amylase. It also relates to a polynucleotide encoding such variants and to the use of the variants in the production of ethanol, beer, dough, maltose syrup and baked products.

### BACKGROUND OF THE INVENTION

Maltogenic alpha-amylase (EC 3.2.1.1) is known to be useful, e.g., for production of ethanol from granular starch by fermentation (WO 2003068976) and for retarding the staling of bread (WO 9104669). One maltogenic alpha-amylase is the commercial product Novamyl® described in EP 120693 B1. Variants of Novamyl are known from WO 9943794. Maltogenic alpha-amylases are known to hydrolyze starch with formation of maltose as the main product together with a minor amount glucose.

### SUMMARY OF THE INVENTION

The inventors realized that in some applications the control of the maltose-to-glucose ratio is of great importance. Particularly for ethanol production from granular starch by fermentation, it may be an advantage to form a larger amount of glucose which is more readily fermentable than maltose. Particularly for production of maltose syrups glucose is an undesired product, and hence it of interest to increase the maltose-to-glucose ratio. They then developed a method of constructing such variants of based on the three-dimensional structure of a parent maltogenic alpha-amylase.

Accordingly, the invention provides a method of constructing a variant polypeptide, comprising:

a) providing a parent maltogenic alpha-amylase having an amino acid sequence and a three-dimensional structure which includes a cleavage point and a substrate with at least three monosaccharide moieties at the reducing side of the cleavage point,

b) selecting an amino acid residue having a C-alpha atom located <10 Å from an atom in the substrate,

c) substituting or deleting the selected residue to obtain a modified amino acid sequence,

d) preparing a polypeptide having the modified sequence,

e) testing the modified polypeptide by incubating it with starch and analyzing the reaction product, and

f) selecting a modified polypeptide which has the ability to hydrolyze starch and wherein the hydrolysis product has a modified maltose-to-glucose ratio compared to an hydrolysis product made with the parent maltogenic alpha-amylase.

The parent maltogenic alpha-amylase and the substrate may for the purpose of steps a), b), and c) be provided in the form of a computer model.

The invention also provides a variant polypeptide which

a) has an amino acid sequence having more than 80% identity to SEQ ID NO: 1,

b) compared to SEQ ID NO: 1 has a different amino acid residue at a position corresponding to W93, T134, G172, N176, D178, F188, D190, D198, I227 V230, K231, H232, F233, Y258, G259, D260, D261, P262, T264, N266, F284, T288 or M330 or a deletion corresponding to 191-195, and

c) has the ability to hydrolyze starch to form an product having a modified maltose-to-glucose ratio than a product made with the polypeptide of SEQ ID NO: 1.

Finally, the invention provides a polynucleotide encoding the polypeptide and uses of the polypeptide in production of ethanol from granular starch by fermentation, in production of maltose syrup, and in the production of dough and baked products.

### DETAILED DESCRIPTION OF THE INVENTION

Maltogenic Alpha-Amylase

The maltogenic alpha-amylase (EC 3.2.1.133) may have the amino acid sequence shown in SEQ ID NO: 1 (in the following referred to as Novamyl) with a 3D structure including a substrate as described in U.S. Pat. No. 6,162,628 and found in the Protein Data Bank with the identifier 1QHO. Alternatively, the maltogenic alpha-amylase may be a Novamyl variant described in U.S. Pat. No. 6,162,628. A 3D structure of such a variant may be developed from the Novamyl structure by known methods, e.g. as described in T. L. Blundell et al., Nature, vol. 326, p. 347 ff (26 Mar. 1987); J. Greer, Proteins: Structure, Function and Genetics, 7:317-334 (1990); or Example 1 of WO 9623874.

Selection of Residues

An amino acid residue is selected which has a C-alpha atom located <10 Å from an atom of the substrate. In 1qho, the following residues are selected by this criterion: 13, 15, 18, 43-44, 70, 72-73, 77-78, 82, 86-94, 97, 127-136, 143, 174-180, 183-184, 187-198, 226-233, 255-267, 270, 282-289, 291-292, 299, 307, 324, 327-331, 360, 370-376.

The selection may in particular be for residues <10 Å from an atom in monosaccharide (glucose) moieties +1, +2 and +3 at the reducing side of the cleavage point. In 1qho, the moieties are denoted j, k and l, and this lead to selection of the following residues: 13, 70, 73, 90, 92-93, 127-132, 174-180, 183-184, 187-191, 196, 226-233, 255-267, 270, 282-289, 291-292, 299, 307, 324, 327-331, 371-372, 375-376.

Amino Acid Substitutions

The selected residue may be substituted so as to push the substrate away or block for it presents in position +1, +2 and +3 etc by making the residues larger at a position corresponding to G172, D178, T189, K231, H232, Y258, G259, D260, T264, N266 or T288 in No-vamyl (SEQ ID NO: 1), e.g. a substitution corresponding to G172V, T189M, K231R, H232Y, Y258W, G259A/H/Y, T264Y/Q/F, N266Y or T288Y/Q/F/P.

The substitution may serve to remove hydrogen bonding or van der Waals contact to the substrate at position +1, +2 and +3. This may be done by substituting with a smaller residue at a position corresponding to W93, T134, D178, D190, D198, 1227, K231, H232, F233, Y258, D260, D261, T264 or T288 of SEQ ID NO: 1, particularly a substitution corresponding to W93S/G/V/T/M/E, T134A, D178L/M/T/V, D190G, D198G, I227V, K231L/M, H232L/M, F233S, Y258L/M/T/V, D260L/M/T/V, D261 G, T264A/V or T288A/V.

Alternatively, a hydrophilic or electrically charged (positive or negative) residue may be substituted with a hydrophobic residue, particularly at a position corresponding to T134, D178, D190, D198, K231, H232 or D261, more particularly a substitution corresponding to T134A, D178V, D190G, D198G, K231 L/M, H232L/M or D261G.

Finally, the substitution or deletion may serve to change indirectly the contact by changing the residues next to the substrate contact residues, particularly a residue corresponding to W93, N176, 191, 192, 193, 194, 195, V230, P262, F284 or M330 in Novamyl, e.g. a substitution corresponding to W93E/G/M/V/T/S, N176L, V230G, F284Y or M330I or a deletion of residues corresponding to 191, 192, 193, 194, and 195.

Amino acid residues are ranked as follows from smallest to largest: (an equal sign indicates residues with sizes that are practically indistinguishable):

G<A=S=C<V=T<P<L=I=N=D=M<E=Q<K<H<R<F<Y<W

The following amino acid residues are considered to be hydrophobic: G, A, V, L, I, P, F, W and C as part of a disulfide bridge.

Variants

Some particular variants according to the invention have the sequence of SEQ ID NO: 1 with the following substitutions:

W93M
W93E
W93M, V230G
Y258W
Y258W, F284Y
H232M
F188T
F188G
F188V
W93G
W93V
W93T
W93S
N176L
D178V
F188G, W93M
F188G, W93E
F188G, W93S
F188G, W93T
F188V, W93M
F188V, W93E
F188V, W93S
F188V, W93T

Ability to Hydrolyze Starch

The variant of the invention is able to hydrolyze starch to form a product having a modified maltose-to-glucose ratio as compared to a product made with the polypeptide of SEQ ID NO: 1. The starch hydrolysis may be carried out by the following procedures described in the examples. The variants of the invention may show an increased ratio of glucose to maltose (DP1/DP2) or an increased ratio of DP1/(DP1-4) or an increased ratio of maltose to glucose (DP2/DP1) or an increased ratio of (DP1-4)/DP1.

Starch is in the context of the present invention intended to include starch as well as breakdown products of starch, such as amylopectin, or amylose, or maltooligosaccharides.

Amino Acid Identity

The polypeptide of the invention may have identities to the disclosed sequences of at least 80%, particularly at least 85% or at least 90%, e.g. at least 91%, or 92%, or 93%, or 94%, or at least 95%, such as 96%, or 97%, or 98%, or 99%.

For purposes of the present invention, alignments of sequences and calculation of identity scores may be done using a Needleman-Wunsch alignment (i.e. global alignment), useful for both protein and DNA alignments. The default scoring matrices BLOSUM50 and the identity matrix are used for protein and DNA alignments respectively. The penalty for the first residue in a gap is −12 for proteins and −16 for DNA, while the penalty for additional residues in a gap is −2 for proteins and −4 for DNA. Alignment is from the FASTA package version v20u6 (W. R. Pearson and D. J. Lipman (1988), “Improved Tools for Biological Sequence Analysis”, PNAS 85:2444-2448, and W. R. Pearson (1990) “Rapid and Sensitive Sequence Comparison with FASTP and FASTA”, Methods in Enzymology, 183:63-98).

Industrial Uses

The variant of the invention may be used in various known applications for amylases, e.g. production of ethanol, beer, dough, maltose syrup and baked products.

Ethanol Production

The variant may be used in a process comprising treating granular starch with the variant and fermentation into ethanol. The treatment of the granular starch serves to produce a hydrolysis product which includes a significant amount of glucose. The fermentation to produce ethanol may be simultaneous with the granular starch treatment, or the starch may first be hydrolyzed followed by fermentation of the hydrolysate. The process may be performed as described in WO 2003068976.

Beer Production

The variant may be used in mashing, i.e. in the process of converting starch from milled malt and solid adjuncts into fermentable and unfermentable sugars to produce wort. The mashing involves incubating the variant with milled malt and solid adjuncts in water to hydrolyze the starch.

Dough and Baked Products

The variant may be added to dough for making baked products such as bread. Addition of the variant may serve to retard staling of the baked product. The addition to dough may be done as described in WO 9104669.

Maltose Syrup

The variant may be used for commercial production of maltose, which today starts from liquefied starch (DE<10), which is subsequently treated simultaneously with debranching enzymes (pullulanase or isoamylase) and maltose-forming enzymes (maltogenic α-amylase or β-amylase) at a temperature around 60° C. Glucose is an undesired side product in maltose syrups because it impacts the crystallization of maltose. Maltose is used in large quantities as syrups in e.g. the confectionary industry and as a sweetening agent in the food industry. Maltose syrups have among other capacities reduced browning capacity, a resistance to moisture absorption and to crystallization making maltose syrups suited for e.g. frozen dessert formulations, hard candy, jams, and jellies. Thus, a maltogenic alpha-amylase with an increased maltose-to-glucose ratio would be an advantage in the production of maltose syrups.

### EXAMPLES

### Example 1

### Starch Hydrolysis with Variants

A number of variants were prepared, each having the sequence of SEQ ID NO: 1 with the indicated substitutions. Each variant was tested by incubating it with maltodextrin (DE 11) by application of the following procedure:
Prepare a 30% (w/w) maltodextrin solution (DE 11) in 50 mM Na-acetate, 1 mM CaCl2 pH 5.5. Is heated to 60° C. for dissolving the maltodextrins.
1 ml substrate is added to 1.5 ml tubes with lid and membrane, and samples are pre-heated to 60° C. on a thermomixer.
1-100 microliter fermentation broth was added to 1 ml preheated substrate. The fermentation broth volumes were adjusted thus, that they all contained the same amount of amylase activity measured by the Phadebas amylase assay.
Samples are incubated for 42 h at 60° C.
Make a small hole in the lid with e.g. a needle.
Samples are boiled for 15 minutes (or 99° C. at the thermomixer).
Add 1 ml Milli-Q.
After cooling the samples are filtered through a 0.2 micro-m filter.

The carbohydrate profile was determined by chromatography by applying standard procedures, e.g. as described in Norman, B. E. in James N. Bemiller, David J. Manners, and Robert J. Sturgeon (eds), Methods in Carbohydrate Chemistry, Volume X. John Wiley & Sons, Inc., New York, pp. 231-239, 1994.

Novamyl without substitutions was included as reference. The results were as follows:

| | | | Maltose/ |
|---|---|---|---|
| | | | glucose |
| Substitutions | % glucose | % maltose | ratio |
| Novamyl parent | 4-5 | 50-55 | 11 |
| W93M | 10 | 42 | 4.2 |
| W93E | 10 | 33 | 3.3 |
| W93M, V230G | 12 | 42 | 3.5 |
| Y258W | 8 | 45 | 5.6 |
| Y258W, F284Y | 8 | 22 | 2.8 |
| H232M | 6 | 22 | 3.7 |
| F188T | 20 | 51 | 2.6 |
| F188G | 20 | 44 | 2.2 |
| F188V | 15 | 55 | 3.7 |
| W93G | 13 | 52 | 4.0 |
| W93V | 13 | 36 | 2.8 |
| W93T | 12 | 37 | 3.1 |
| W93S | 8 | 36 | 4.5 |
| W93T, F188V | 14 | 27 | 1.9 |
| N176L | 11 | 50 | 4.5 |
| D178V | 12 | 51 | 4.3 |
| N26S, L51M, T80A, F237L, N266Y, | 10 | 42 | 4.2 |
| M330I | | | |
| d(191-195)¹⁾, D261G, T288P | 8 | 21 | 2.6 |
| W185R, D198G, E202V | 16 | 52 | 3.3 |
| T134A, H170R, D190G, V215A, | 4 | 14 | 3.5 |
| F233S, I251T | | | |
| G172V, D178V, G204D | 11 | 45 | 4.1 |
| R55C, K137M, 288S, S331P, 396V | 4.5 | 52.5 | 11.7 |
| N176Y, E202D | 3.5 | 43.1 | 12.3 |
| T189M, A219V | 4.1 | 49.8 | 12.1 |
| T189M, A214T, F237L, T288S | 3.2 | 41.6 | 13.1 |
| D161G, N176Y, T189M, N203D, | 3.4 | 45.4 | 13.3 |
| A214T | | | |
| A148D, T189M, A219V | 3.6 | 47.2 | 13.1 |
| T189M, Q208R, A219V, D657G | 4.0 | 50.2 | 12.5 |
| F104L, N106D, K137M, D173N, | 3.3 | 54.7 | 16.7 |
| N176Y, T189M, E202D, | | | |
| V254A, L334, P380L, | | | |
| G512D, Y632C | | | |
| K137M, T189M, S195T, E202D, | 3.7 | 57.8 | 15.5 |
| G263R, S331P, A388V, | | | |
| N631S | | | |
| H103R, T189M, I227V, K239R, | 2.4 | 38.8 | 15.9 |
| V254A, T288S, S441P, | | | |
| Y460H, F649L | | | |

¹⁾d (191-195) indicates a deletion of the amino acids corresponding to position 191, 192, 193, 194, and 195.

### Example 2

### Starch Hydrolysis with Variants and Pullulanase

Further a number of variants were tested applying the same procedure as described in Example 1, except that 1.2 mg/g DS of the commercially available pullulanase Promozyme® (EP 63909) was added.

The following results were obtained:

| | | | Maltose/ |
|---|---|---|---|
| | | | glucose |
| Substitutions | % glucose | % maltose | ratio |
| Parent Novamyl | 7.2 | 71.6 | 9.9 |
| A148D, T189M, G263R, N337D, | 5.9 | 69.8 | 11.9 |
| Y572C, F636L | | | |
| D173N, N176Y, T189M, A219V, | 5.5 | 70.6 | 12.8 |
| Y246H, T288S, L334P, | | | |
| N631S, K650R | | | |
| N27S, T80I, T189M, S195T, E202D, | 4.9 | 67.6 | 13.7 |
| I290V, T386A, L596P | | | |
| A148D, T189M, D212G, A219V, | 5.9 | 69.9 | 11.9 |
| T288S | | | |
| K137M, N158Y, N176H, T189M, | 5.1 | 63.8 | 12.4 |
| E202D, V254A, S331P, A388V | | | |

### Example 3

### Starch Hydrolysis with Purified Variants and Pullulanase

A number of purified variants (each having the sequence of SEQ ID NO: 1 with the indicated substitutions) were prepared by standard purification techniques, see e.g. Beier et al.: “Conversion of the maltogenic alpha-amylase Novamyl into a CGTase” in Protein Engineering, vol. 13 no. 7 pp. 509-513, 2000.

Each variant was tested by incubating it with maltodextrin (DE 11) at 60° C. and pH 5.5 for 42 hours as described in Example 1. Either an amount of 0.81 micro g (variants marked with [1]) or 1.62 micro g (variants marked with [2]) of the variant was added, and further 1.2 mg/g DS of the commercially available pullulanase Promozyme® (EP 63909) was added.

Novamyl without substitutions was included as reference. The results were as follows:

| | | | Maltose/ |
|---|---|---|---|
| | | | glucose |
| Substitutions | % maltose | % glucose | ratio |
| Novamyl parent [1] | 56.7 | 5.5 | 10.3 |
| Novamyl parent [2] | 66.1 | 6.6 | 10.1 |
| Y258W [1] | 32.0 | 5.5 | 5.8 |
| Y258W [2] | 37.3 | 6.8 | 5.5 |
| W93S [1] | 40.4 | 11.2 | 3.6 |
| W93S [2] | 46.0 | 14.2 | 3.2 |
| T189M, A214T, F237L, T288S [1] | 56.6 | 5.0 | 11.3 |
| T189M, A214T, F237L, T288S [2] | 63.0 | 5.8 | 10.9 |
| D161G, N176Y, T189M, N203D, | 48.1 | 3.8 | 12.7 |
| A214T [1] | | | |
| D161G, N176Y, T189M, N203D, | 61.3 | 5.2 | 11.8 |
| A214T [2] | | | |
| A148D, T189M, A219V [1] | 54.6 | 4.4 | 12.4 |
| A148D, T189M, A219V [2] | 63.4 | 5.4 | 11.7 |
| T189M, Q208R, A219V, D657G [1] | 52.8 | 4.4 | 12.0 |
| T189M, Q208R, A219V, D657G [2] | 65.7 | 5.9 | 11.1 |
| F104L, N106D, K137M, | 35.0 | 2.0 | 17.5 |
| D173N, N176Y, T189M, | | | |
| E202D, V254A, L334P, | | | |
| P380L, G512D, Y632C [1] | | | |
| F104L, N106D, K137M, D173N, | 40.6 | 2.3 | 17.7 |
| N176Y, T189M, E202D, V254A, | | | |
| L334P, P380L, G512D, | | | |
| Y632C [2] | | | |
| K137M, T189M, S195T, E202D, | 59.9 | 3.8 | 15.8 |
| G263R, S331P, A388V, | | | |
| N631S [1] | | | |
| K137M, T189M, S195T, E202D, | 67.9 | 4.6 | 14.8 |
| G263R, S331P, A388V, | | | |
| N631S [2] | | | |
| H103R, T189M, I227V, K239R, | 36.7 | 2.5 | 15.0 |
| V254A, T288S, S441P, | | | |
| Y460H, F649L [1] | | | |
| H103R, T189M, I227V, K239R, | 68.2 | 4.8 | 14.2 |
| V254A, T288S, S441P, | | | |
| Y460H, F649L [2] | | | |

### Example 4

### Starch Hydrolysis with Purified Variants without Addition of Pullulanase

A number of purified variants (each having the sequence of SEQ ID NO: 1 with the indicated substitutions) were prepared by standard purification techniques, see e.g. Beier et al.: “Conversion of the maltogenic alpha-amylase Novamyl into a CGTase” in Protein Engineering, vol. 13 no. 7 pp. 509-513, 2000.

Each variant was tested by incubating it with maltodextrin (DE 11) at 60° C. and pH 5.5 for 42 hours as described in Example 1. An amount 1.62 micro g (variants marked with [2]) of the variant was added.

A single variant was dosed at a higher amount, namely 38.2 micro g (variant marked with [3]).

| | | | Maltose/ |
|---|---|---|---|
| | | | glucose |
| Substitutions | % maltose | % glucose | ratio |
| Parent Novamyl [2] | 57.8 | 5.2 | 11.0 |
| F188G [3] | 46.2 | 26.3 | 1.8 |
| T189M, A214T, F237L, T288S [2] | 53.4 | 4.4 | 12.1 |
| D161G, N176Y, T189M, | 57.7 | 4.7 | 12.3 |
| N203D, A214T [2] | | | |
| A148D, T189M, A219V [2] | 56.4 | 4.4 | 12.7 |
| T189M, Q208R, A219V, D657G [2] | 55.5 | 4.5 | 12.3 |

### Example 5

### Amylopectin Hydrolysis with Variants

A number of purified variants (each having the sequence of SEQ ID NO: 1 with the indicated substitutions) were prepared by standard purification techniques, see e.g. Beier et al.: “Conversion of the maltogenic alpha-amylase Novamyl into a CGTase” in Protein Engineering, vol. 13 no. 7 pp. 509-513, 2000.

Each variant was tested by incubating it with amylopectin (waxy maize starch) by application of the following procedure:
Prepare a 5% (w/w) amylopectin solution in 50 mM Na-acetate, 1 mM CaCl₂, pH 5.5. The solution is boiled for 2 minutes or until it is dissolved.
1 mL substrate is added to 1.5 mL tubes with lid and membrane, and samples are preheated to 60° C. on a thermomixer.
Enzyme is dosed at a dose corresponding to the amylase activity of 0.81 micro g No-vamyl measured by the Phadesbas amylase assay (which is 0.045 PSU/mL substrate, when Novamyl activity is 56.8PSU/mg)
Samples are incubated for 1 hour or 24 hours at 60° C.
1 ml Milli-Q is added with 1-2 drops of 1 M HCl (pH must be less than 3 to inactive the amylase).
Make a small hole in the lid with e.g. a needle.
Samples are boiled for 15 minutes.
After cooling the samples are filtered through a 0.2 micro m filter.
   Samples incubated for 1 hour are marked [1], samples incubated for 24 hours are marked [24].

The carbohydrate profile was determined by chromatography by applying standard procedures, e.g. as described in Norman, B. E. in James N. Bemiller, David J. Manners, and Robert J. Sturgeon (eds), Methods in Carbohydrate Chemistry, Volume X. John Wiley & Sons, Inc., New York, pp. 231-239, 1994.

Novamyl without substitutions was included as reference. The results were as follows:

| | | | Maltose/ |
|---|---|---|---|
| | | | glucose |
| Substitutions | % maltose | % glucose | ratio |
| Novamyl parent [1] | 53.2 | 1.5 | 35.1 |
| Novamyl parent [24] | 64.3 | 2.6 | 24.7 |
| Y258W [1] | 39.1 | 2.7 | 14.4 |
| Y258W [24] | 57.6 | 4.1 | 13.9 |
| F188G [1] | 12.3 | 0.0 | — |
| F188G [24] | 17.4 | 2.4 | 7.2 |
| W93S [1] | 13.3 | 1.8 | 7.5 |
| W93S [24] | 52.7 | 15.3 | 3.4 |
| F104L, N106D, K137M, D173N, | 49.9 | 1.2 | 41.6 |
| N176Y, T189M, E202D, V254A, | | | |
| L334P, P380L, G512D, Y632C [1] | | | |
| F104L, N106D, K137M, D173N, | 66.9 | 1.5 | 44.6 |
| N176Y, T189M, E202D, V254A, | | | |
| L334P, P380L, G512D, Y632C [24] | | | |
| K137M, T189M, S195T, E202D, | 66.2 | 0.6 | 110 |
| G263R, S331P, A388V, N631S [1] | | | |
| K137M, T189M, S195T, E202D, | 64.4 | 0.8 | 80.5 |
| G263R, S331P, A388V, N631S | | | |
| [24] | | | |
| H103R, T189M, I227V, K239R, | 54.0 | 0.9 | 60.0 |
| V254A, T288S, S441P, Y460H, | | | |
| F649L [1] | | | |
| H103R, T189M, I227V, K239R, | 63.0 | 1.3 | 48.5 |
| V254A, T288S, S441P, Y460H, | | | |
| F649L [24] | | | |

### Example 6

### Baking with Variants

Two variants were tested for baking, namely Y258W and W93S. Bread was made by the European Straight Dough method with and without addition of enzymes. The texture was evaluated using standard AACC procedures, and the following results were obtained after 7 days storage:

| | | |
|---|---|---|
| Reference: no enzyme | Y258W | W93S |

Firmness (g)

| | | |
|---|---|---|
| 1600 | 1250 | 1250 |

Elasticity % (g/g)

| | | |
|---|---|---|
| 50 | 53 | 54 |

Free water mobility (micro S)

| | | |
|---|---|---|
| 10600 | 10850 | 11100 |

Y258W was dosed 5 mg enzyme protein/kg flour for all three tests.
W93S was dosed 3 mg enzyme protein/kg flour for all three tests.

1-14. (canceled)

15. A method of constructing a polypeptide, comprising:
a) providing a parent maltogenic alpha-amylase having an amino acid sequence and a three-dimensional structure which includes a cleavage point and a substrate with at least three monosaccharide moieties at the reducing side of the cleavage point,
b) selecting an amino acid residue having a C-alpha atom located <10 Å from an atom in the substrate,
c) substituting or deleting the selected residue to obtain a modified amino acid sequence,
d) preparing a polypeptide having the modified sequence,
e) testing the modified polypeptide by incubating it with starch and analyzing the reaction product, and
f) selecting a modified polypeptide which has the ability to hydrolyze starch and wherein the hydrolysis product has a modified maltose-to-glucose ratio compared to a hydrolysis product made with the parent maltogenic alpha-amylase.

16. The method of claim 15, wherein the selected residue has a C-alpha atom located <10 Å from an atom in the three monosaccharide moieties at the reducing side of the cleavage point.

17. The method of claim 15, wherein the substitution is with a larger residue.

18. The method of claim 15, wherein the substitution is with a larger residue at a position corresponding to Y258, G172, D178, T189, K231, H232, G259, D260, T264, N266 or T288 of SEQ ID NO: 1.

19. The method of claim 15, wherein the substitution is a substitution corresponding to Y258W, G172V, T189M, K231R, H232Y, G259A/H/Y, T264Y/Q/F, N266Y or T288Y/Q/F/P.

20. The method of claim 15, wherein the substitution is with a smaller residue.

21. The method of claim 15, wherein the substitution is with a smaller residue at a position corresponding to W93, T134, D178, D190, D198, I227, K231, H232, F233 Y258, D260, D261, T264 or T288 of SEQ ID NO: 1.

22. The method of claim 15, wherein the substitution is a substitution corresponding to W93S/G/V/T/M/E, T134A, D178L/M/T/V, D190G, D198G, I227V, K231L/M, H232L/M, F233S, Y258L/M/T/V, D260L/M/T/V, D261G, T264A/V, or T288A/V.

23. The method of claim 15, wherein the substitution is with a hydrophobic residue.

24. The method of claim 15, wherein the substitution is with a hydrophobic residue at a position corresponding to T134, D178, D190, D198, K231; H232 or D261.

25. The method of claim 15, wherein the substitution is a substitution corresponding to T134A, D178V, D190G, D198G, K231L/M, H232L/M or D261G.

26. The method of claim 15, wherein the selected residue corresponds to W93, N176, 191, 192, 193, 194, 195, V230, P262, F284 or M330 of SEQ ID NO: 1.

27. The method of claim 15, wherein the substitution corresponds to W93E/G/M/V/T/S, N176L, V230G, F284Y or M330I or the deletion is of residues corresponding to 191-195.

28. A polypeptide which
a) has an amino acid sequence having more than 80% identity to SEQ ID NO: 1,
b) compared to SEQ ID NO: 1 has a different amino acid residue at a position corresponding to W93, T134, G172, N176, D178, F188, D190, D198, I227, V230, K231, H232, F233, Y258, G259, D260, D261, P262, T264, N266, F284, T288 or M330 or a deletion corresponding to 191-195, and
c) has the ability to hydrolyze starch to form an product having a modified maltose-to-glucose ratio than a product made with the polypeptide of SEQ ID NO: 1.

29. The polypeptide of claim 28, which compared to SEQ ID NO: 1 comprises a substitution corresponding to W93E/G/M/V/T/S, T134A, G172V, D178L/M/T/V, F188G/T/V, D190G, D198G, I227V, V230G, K231R/L/M, 232Y/L/M, F233S, Y258W/L/M/T/V, G259A/H/Y, D260L/M/T/V, D261G, T264Y/Q/F/A/V, N266Y, F284Y, T288Y/Q/F/A/V/P or M330I.

30. The polypeptide of claim 28, which has the amino acid sequence of SEQ ID NO: 1 with the following alterations:
W93M
W93E
W93M, V230G
Y258W
Y258W, F284Y
H232M
F188T
F188G
F188V
W93G
W93V
W93T
W93S
N176L
D178V
F188G, W93M
F188G, W93E
F188G, W93S
F188G, W93T
F188V, W93M
F188V, W93E
F188V, W93S
F188V, W93T

31. A polynucleotide encoding the polypeptide of claim 28.

32. A dough comprising the polypeptide of claim 28.

33. A method of producing dough or a baked product from dough, comprising adding the polypeptide of claim 28 to the dough or baked product.

34. A process for the production of maltose syrup comprising treating liquefied starch with the polypeptide of claim 28.


SEQUENCE LISTING (1 sequence)

SEQ ID NO: 1 - protein, 686 aa, Bacillus stearothermophilus
SSSASVKGDVIYQIIIDRFYDGDTTNNNPAKSYGLYDPTKSKWKMYWGGDLEGVRQKLPYLKQLGVTTIWLSPVLDNLDTLAGTDNTGYHGYWTRDFKQIEEHFGNWTTFDTLVNDAHQNGIKVIVDFVPNHSTPFKANDSTFAEGGALYNNGTYMGNYFDDATKGYFHHNGDISNWDDRYEAQWKNFTDPAGFSLADLSQENGTIAQYLTDAAVQLVAHGADGLRIDAVKHFNSGFSKSLADKLYQKKDIFLVGEWYGDDPGTANHLEKVRYANNSGVNVLDFDLNTVIRNVFGTFTQTMYDLNNMVNQTGNEYKYKENLITFIDNHDMSRFLSVNSNKANLHQALAFILTSRGTPSIYYGTEQYMAGGNDPYNRGMMPAFDTTTTAFKEVSTLAGLRRNNAAIQYGTTTQRWINNDVYIYERKFFNDVVLVAINRNTQSSYSISGLQTALPNGSYADYLSGLLGGNGISVSNGSVASFTLAPGAVSVWQYSTSASAPQIGSVAPNMGIPGNVVTIDGKGFGTTQGTVTFGGVTATVKSWTSNRIEVYVPNMAAGLTDVKVTAGGVSSNLYSYNILSGTQTSVVFTVKSAPPTNLGDKIYLTGNIPELGNWSTDTSGAVNNAQGPLLAPNYPDWFYVFSVPAGKTIQFKFFIKRADGTIQWENGSNHVATTPTGATGNITVTWQN